(19) United States
(12) Patent Application Publication
(10) Pub. No.: US 2007/0161922 A1
(43) Pub. Date: Jul. 12, 2007

(54) Method of infrared tomography, active and passive, for earlier diagnosis of breast cancer

(75) Inventors: Ben Zion Dekel, Hadera (IL); Nathan Blaunshtein, Beer Sheva (IL); Avaraham Yarkony, Beer Sheva (IL); Arkadii Zilberman, Beer Sheva (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Medical Optical Imaging Systems Ltd., Tel Aviv (IL)

(21) Appl. No.: 11/535,105

(22) Filed: Sep. 26, 2006

Related U.S. Application Data
(60) Provisional application No. 60/757,006, filed on Jan. 9, 2006.

(51) Int. Cl. A61B 5/00

(52) U.S. Cl. 600/549

(57) ABSTRACT

A device and method are disclosed to non-invasively identify a lesion inside a region of living tissue. The region is exposed to medium infrared (MIR) radiation to preferentially heat the lesion The region is then scanned for black body radiation in a medium infrared waveband A lesion, being hotter than the surrounding tissue, is detected as domain of increased local emittance of MIR radiation Further scanning or heating in a second waveband is used to identify a particular class of lesions. The invention is particularly useful for early identification of malignant breast cancer.

This is a continuation-in-part of U.S. Provisional Patent Application No. 60/757006, filed Jan. 9, 2006.

### FIELD AND BACKGROUND OF THE INVENTION

The present invention relates to a non-invasive method and device to identify anomalous structures inside living tissue. More specifically the present invention relates to a method and device for non-intrusive detection and identification of different lesions and particularly of breast cancers by combined passive and active analyses of infra-red optical signals based on integral and spectral regimes for detection and imaging leading to earlier warning and treatment of potentially dangerous conditions.

According to current practice suspicious lesions are commonly biopsied to determine their status. Biopsies have many obvious disadvantages: firstly biopsies require intrusive removal of tissue that can be painful and expensive. Only a very limited number of sights can be biopsied in one session and patients are not likely to put up with a large number of such expensive painful tests. Furthermore, biopsy samples must be stored and transported to a laboratory for expert analysis. Storage and transportation increase the cost, increases the possibility that samples will be mishandled, destroyed or lost, and also causes a significant time delay in receiving results. This time delay means that examination follow up requires bringing the patient back to the doctor for a separate session. This increases the inconvenience to the patient, the cost and the risk that contact will be lost or the disease will precede to a point of being untreatable. Furthermore, the waiting period causes significant anxiety to the patient. Finally, interpretation of biopsies is usually by microscopic analysis producing qualitative subjective results, which may lead to ambiguous inconsistent interpretation.

Therefore, in medical diagnosis there is great interest in safe, non-intrusive detection technologies, particularly, in the case of cancer. Cancer is a disease that develops slowly and can be prevented by monitoring lesions with potential to become cancerous through routine screening. There is, nevertheless, a limit to the amount of time, money or inconvenience that a basically healthy patient is willing to dedicate to routine screening procedures. Therefore, screening must be able to reliably identify dangerous tumors and differentiate dangerous tumors from benign conditions quickly, inexpensively and safely.

There are many methods for spectral analysis and imaging of tissue anomalies using active regimes, which are widely known. These methods include optical spectral and thermal imaging methods in the visible (VIS) and infrared (IR) wavebands, as well as electromagnetic microwave, acoustic, magnetic resonance imaging (MRI), magnetic resonance spectrum (MRS), ultraviolet (UV) and X-ray methods [see for example Fear, E. C., and M. A. Stuchly, “Microwave detection of breast tumors: comparison of skin subtraction algorithms”, SPIE, vol. 4129, 2000, pp. 207-217; R. F. Brem, D. A. Kieper, J. A. Rapelyea and S. Majewski, “Evaluation of a high resolution, breast specific, small field of view gamma camera for the detection of breast cancer”, Nuclear Instruments and Methods in Physics Research, vol. A 497, 2003, pp. 39-45.]

X-ray technology, which has been used successfully for detection of anomalies inside the human-body since the early 60's, is not suited for earlier detection of cancer due to the dangerous effects of X-ray radiation on human health. Particularly x-rays cannot be used for diagnostics of patients who need intensive reexamination over short-time periods.

Acoustic active methodologies, which are useful for detection of structures inside the human body, are also non-effective for early diagnosis of breast cancer. Precancerous lesions are often of microscopic dimensions (on the order of millimeters or micrometers), which cannot be detected and identified by use of acoustic methods (which are limited to detecting structures larger than the wavelength of sound on the order of centimeters).

Microwave detection of tumors is based on the contrast in dielectric properties of normal and anomalous tissues. Microwave technologies are very complicated and radiate the human body with microwave radiation, which may have dangerous effects. Furthermore, microwave signals have wavelength from a few mm to a few cm, and therefore microwaves cannot identify small structures with diameter of half mm or less. Such anomalies, on the half mm scale, are very important in early cancer diagnosis [Bruch, R., et al, “Development of X-ray and extreme ultraviolet (EUV) optical devices for diagnostics and instrumentation for various surface applications”, Surface and Interface Anal. vol. 27, 1999, pp. 236-246].

Magnetic methods (MRI and MRS) provide anatomic images in multiple planes enabling tissue characterization. Contrast enhanced MR studies have been found to be useful in the diagnosis of small tumors in dense breast tissue and in differentiating benign anomalies from malignant ones [U. Sharma, V. Kumar and N. R. Jagannathan, “Role of magnetic resonance imaging (MRI), MR spectroscopy (MRS) and other imaging modalities in breast cancer”, National Academy Science Letters-India, vol. 27, No. 11-12, pp. 373-85, 2004]. In vivo MRS has been used to assess the biochemical status of normal and diseased tissues. These MR methods are very expensive and cannot always distinguish between malignant and benign conditions and can't detect micro-calcifications.

Optical methods for detection, identification and diagnosis of internal abnormalities have been applied in order to avoid the above disadvantages of tradition biopsies and their interpretation. Optical methods can be classified into two regimes. The first is called the integral regime of detection. In the integral regime, the spatial distribution of a signal is measured to obtain information about changes in properties (like temperature or chemical content), which mark the boundaries between normal anomalous domains. The second regime is called the spectral regime. In the spectral regime, radiation intensities are measured in various frequency bands. The spectral regime is useful for identification of specific anomalies based on information about the corresponding “signature” of the anomaly in the frequency domain.

Previous art optical evaluation of internal tissue is based on active illumination with light in the near infrared NIR waveband. The reason that NIR light is preferred is because NIR light is safe and NIR radiation penetrates healthy skin tissue and allows non-intrusive detection anomalous internal structures. Nevertheless, all of the widely known techniques such as optical imaging, optical spectral analysis, and thermal imaging have disadvantages and are not fully appropriate for detection and identification of breast cancer and cancer precursors.

The fluorescent method is based on illumination of the suspected area with a UV light source and detection of the fluorescence spectrum in the NIR/VIS range. Malignant tumors can be identified due to differences in auto fluorescence spectra between normal tissue and cancerous tissue [Y. Chen, X. Intes and B. Chance, “Development of high-sensitivity neat-infrared fluorescence imaging device for early cancer detection”, Biomedical Instrumentation & Technology, vol. 39, No. 1, pp. 75-85, 2005]. A major problem using auto fluorescence for early cancer detection is that auto fluorescence of cancerous lesions produces a weak signal over a wide waveband including wavelengths that are strongly dispersed and confounded by other signals from various chemicals found in human tissue. Due to this dispersion, auto flourescence imaging does produce a clear focused image of a specific anomaly. Also detection of weak auto flourescence signals is very expensive.

In order to produce stronger, sharper NIR fluorescence images, Licha et al. 2006 [U.S. Pat. No. 7,025,949] have suggested injecting a fluorescent dye into a patient. The dye is engineered such that it accumulates in cancerous tissue and produces a strong narrow band fluorescence signal that can more easily and more precisely be detected and located. The use of dyes has obvious disadvantages. Engineered dyes are expensive. Furthermore, injecting dye into a patient is intrusive and inconvenient. Therefore, patients are likely to resist the injection of dyes for routine diagnostic procedures.

The photon migration method is another noninvasive clinical technique based on measuring the absorption and scattering of a few wavelengths of NIR radiation by breast tissue [Shah, N., A. E. Ceirusi, D. Jakubowski, D. Hsianq, J. Butler and B. J. Tromberq, “Spatial variations in optical and physiological properties of healthy breast tissue”, Journal of Biomedical Optics, vol. 9, No. 3, 2004, pp. 534-40]. Photo migration measurements allow determination of oxy and deoxy hemoglobin, lipid and water concentration. Characteristic differences in these concentrations between healthy and diseased tissue indicate a lesion. All of the above NIR techniques require expensive technology to detect photon migration and scattering. Furthermore, none of the NIR methodologies can differentiate between malignant and benign lesions. Thus, NIR methods produce a large number a false positive results causing worry to patients and requiring invasive screening.

Narrow band medium infrared (MIR) methodologies for analyzing and classifying pathologies include Raman spectroscopy and methods based on MIR spectroscopic diagnostics (called Fourier-transform-infrared spectroscopy, FTIR), which can be combined with fiber optic techniques (called fiber-optical evanescent wave method, FEW) [Afanasyeva, N., S. Kolyakov, V. Letokhov, et al, “Diagnostic of cancer by fiber optic evanescent wave FTIR (FEW-FTIR) spectroscopy”, SPIE, vol. 2928, 1996, pp. 154-157; Afanasyeva, N., S. Kolyakov, V. Letokhov, et al, “Noninvasive diagnostics of human tissue in vivo”, SPIE, vol. 3195, 1997, pp. 314-322; Afanasyeva, N., V. Artjushenk, S. Kolyakov, et al., “Spectral diagnostics of tumor tissues by fiber optic infrared spectroscopy method”, Reports of Academy of Science of USSR, vol. 356, 1997, pp. 118-121; Afanasyeva, N., S. Kolyakov, V. Letokhov, and V. Golovidna, “Diagnostics of cancer tissues by fiber optic evanescent wave Fourier transform IR (FEW-FTIR) spectroscopy”, SPIE, vol. 2979, 1997, pp. 478-486; Bruch, R., S. Sukuta, N. I. Afanasyeva, et al., “Fourier transform infrared evanescent wave (FTIR-FEW) spectroscopy of tissues”, SPIE, vol. 2970, 1997, pp. 408-415; Sukuta, S., and R. Bruch, “Factor analysis of cancer Fourier transform evanescent wave fiber-optical (FTIR-FEW) spectra”, Lasers in Surgery and Medicine, vol. 24, No. 5, 1999, pp. 325-329; Afanasyeva, N., L. Welser, R. Bruch, et al., “Numerous applications of fiber optic evanescent wave Fourier transform infrared (FEW-FTIR) spectroscopy for subsurface structural analysis”, SPIE, vol. 3753, 1999, pp. 90-101]. These techniques use a narrow spectral wavebands in the medium infrared range, (e.g. from 3-5 μm or from 10-14 μm) [Artushenko, V., A. Lerman, A. Kryukov, et al., “MIR fiber spectroscopy for minimal invasive diagnostics”, SPIE, vol. 2631, 1995]). These narrow band IR methods are effective for differentiating normal tissue from abnormal tissue. Nevertheless, being limited to measurements of narrow band IR these methods cannot detect subtle differences between a non-pathologic conditions and early cancer precursors and cannot trace the development of lesions from benign to precancerous to malignant.

Current art non-invasive passive MIR methods use thermo and/or FLIR cameras to produce color images of pathological anomalies based on difference in MIR emission from normal and cancerous tissues. These methods have been of great value in detecting and identifying cancer on the body surface (e.g. melanoma and skin cancer). For skin tumors, thermal images provide doctors with four main parameters for each pathological anomaly: a) asymmetry of the cancerous tissue structure shape; b) bordering of the cancerous tissue structure; c) color of the cancerous tissue structure d) dimensions of the cancerous tissue structure. However, these methods are not applicable to the detection of internal lesions such as breast cancer.

FLIR cameras, detect of photons radiated by the human body, as a “black body”, at the waveband from 7 to 13 μm (the waveband for which radiation energy from human body is maximum). In this waveband, there is a lot of noise from background obstructions having similar temperature to the human body, i.e., from 280 K to 320 K. Such background noise makes it impossible, using current technology, to reliably identify weak attenuated passive signals from internal lesions.

The use of thermo cameras, which measure heat flows from human body as a “thermal waves” in the 2 to 5 μm waveband, has the similar drawbacks to those mentioned above for FLIR cameras. Despite the fact that the thermal cameras detect a shorter wavelength band corresponding to higher temperatures (from 350 K to 400 K) than that detected by a FLIR, and therefore, thermal cameras are not seriously affected by background noise. Nevertheless the total intensity of passive “black body” thermal waves radiated from human body in the 2 to 5 μm waveband is too small to be detected after attenuation by intervening tissue for lesions at depths of more than few mm

Thus current art non-invasive methods for passive MIR detection (whether based on FTIR, FEW, or thermal imaging with FLIR's or thermal cameras), which have been of great value in detecting skin cancer, cannot be used for detecting breast cancer at a depth of a centimeter or more beneath the skin surface. At such a depth, the increased radiation intensity due to the slight naturally increase in temperature of tumors compared to healthy tissue (on the order of 0.1° K) is highly attenuated and not detectable with commonly available instruments

Thus, there is a widely recognized need for, and it would be highly advantageous to have, a non-invasive methodology to detect and identify pathologic lesions and particular early cancer precursors at a depth of a few centimeters in living tissue. The current invention fills this need by employing active preferentially heating based on the preferential absorption of MIR radiation by cancerous tissue, as well as a differential measure to improve sensitivity to subtle differences in intensity of MIR emission. This enhanced thermal contrast and improved sensitivity allows precise spectral quantification of changes in light absorption and heat generation that are characteristic of different forms of lesions and stages of cancer development. Therefore the present invention discloses an extremely sensitive non-invasive method to differentiate in-vivo between normal cells and cells having pathological anomalies.

In embodiments described below, the differential measure, contrast, is used to differentiate between the normal cells and cells with pathological anomalies in an integral regime and a spectral regime of analysis. Spatial distribution of contrast over a wide frequency band is taken into account in the integral regime to detect a lesion and to assess the position, size and shape of the lesion. Frequency dependence of the contrast, its magnitude and its sign are used to assess vascular and metabolic activity, which are different for normal tissue and tissue with pathological anomalies. Combined together, both regimes allow precise diagnostics of tissue anomalies and facilitate earlier warning of cancerous and precancerous conditions. As a non-invasive method, the proposed invention reduces the cost, discomfort and danger of cancer screening.

### SUMMARY OF THE INVENTION

The present invention is a non-invasive method and device to identify pathological lesions inside of living tissue. More specifically the present invention relates to a method and device for non-intrusive detection and identification of different kinds of tumors, lesions and cancers (namely, breast cancer) by combined active/passive analyses of infra-red optical signals based on integral and spectral regimes for detection and imaging leading earlier warning and treatment of potentially dangerous conditions.

According to the teachings of the present invention, there is provided a non-intrusive method for identifying an anomalous domain under the skin in a region of a patient. The method includes the steps of heating the anomalous domain preferentially over healthy tissue and measuring a radiation emitted by the anomalous domain due to the domains increased temperature as a result of being heated. The anomalous domain is detected based on a result of the measuring.

According to the teachings of the present invention, there is also provided a detector to reveal an anomalous domain under a skin of a region of a patient. The detector includes a lamp for exposing the skin of the region to MIR radiation, beating the region. Particularly, the MIR radiation preferentially heats the anomalous domain. The detector further includes a timer for turning off the lamp after a predetermined period of exposure. The detector also includes a MIR sensor for measuring a radiation emitted from the region after the lamp is turned off.

According to further features in preferred embodiments of the invention described below, the step of heating includes applying infrared radiation in a first waveband to the region.

According to still further features in the described preferred embodiments, the first waveband differs from the wave band of the measured radiation emitted from the region.

According to still further features in the described preferred embodiments, the method further includes the step of applying an infrared radiation in a second waveband to the region.

According to still further features in the described preferred embodiments, the first waveband includes infrared radiation having a wave number 1600-1700 cm⁻¹.

According to still further features in the described preferred embodiments, the measured emitted radiation includes a black body radiation in a medium infrared waveband.

According to still further features in the described preferred embodiments, the region being scanned includes a portion of the breast of the patient.

According to still further features in the described preferred embodiments, the step of heating continues for a predetermined period of time and the step of measuring occurs after the end of the time of heating.

According to still further features in the described preferred embodiments, the measurement result used to determine the presence of the anomaly is a differential measure of the emitted radiation

According to still further features in the described preferred embodiments, the differential measure is a contrast. The contrast may includes a difference between the radiation intensity in the domain and a background radiation or the contrast may include a difference between the radiation intensity in the domain in a first waveband and the radiation intensity in the domain in a second waveband.

According to still further features in the described preferred embodiments, the method further includes the step of performing spectral analysis to identify the anomalous domain.

According to still further features in the described preferred embodiments, the method further includes the step of determining a depth of the anomalous domain.

According to still further features in the described preferred embodiments, the detector further includes a band pass filter to limit the sensitivity of the sensor to a first narrow waveband.

According to still further features in the described preferred embodiments, the detector includes a second sensor for measuring radiation in a second waveband.

### BRIEF DESCRIPTION OF THE DRAWINGS

The invention is herein described, by way of example only, with reference to the accompanying drawings, where:

FIG. 1 is of a detector according to a first embodiment of the current invention;

FIG. 2 is an MIR absorbance spectrograph of healthy, benign and malignant breast tissue in a first waveband 1500-1800 cm⁻¹ (λ=6-7 μm);

FIG. 3 is an MIR contrast spectrograph of healthy, benign and malignant breast tissue in a first waveband 1500-1800 cm⁻¹ (λ=6-7 μm);

FIG. 4a is an MIR absorbance spectrograph of healthy, benign and malignant breast tissue in a second waveband 900-1200 cm⁻¹ (λ=8-11 μm);

FIG. 4b is an MIR absorbance spectrograph of healthy, benign and malignant breast tissue in a third waveband 1400-1750 cm⁻¹ (λ=6-7 μm);

FIG. 4c is an MIR absorbance spectrograph of healthy, benign and malignant breast tissue in a fourth waveband 2700-3600 cm⁻¹ (λ=3-4 μm);

FIG. 5 is a flowchart illustrating a first embodiment of the current invention;

FIG. 6a illustrates a second embodiment of a device to identify lesions inside living tissue according to the current invention.

FIG. 6b is a flowchart illustrating a second embodiment of the current invention.

### DESCRIPTION OF THE PREFERRED EMBODIMENTS

The principles and operation of a non-invasive method and device to identify pathological skin lesions according to the present invention may be better understood with reference to the drawings and the accompanying description.

It will be appreciated that the above descriptions are intended only to serve as examples, and that many other embodiments are possible within the spirit and the scope of the present invention.

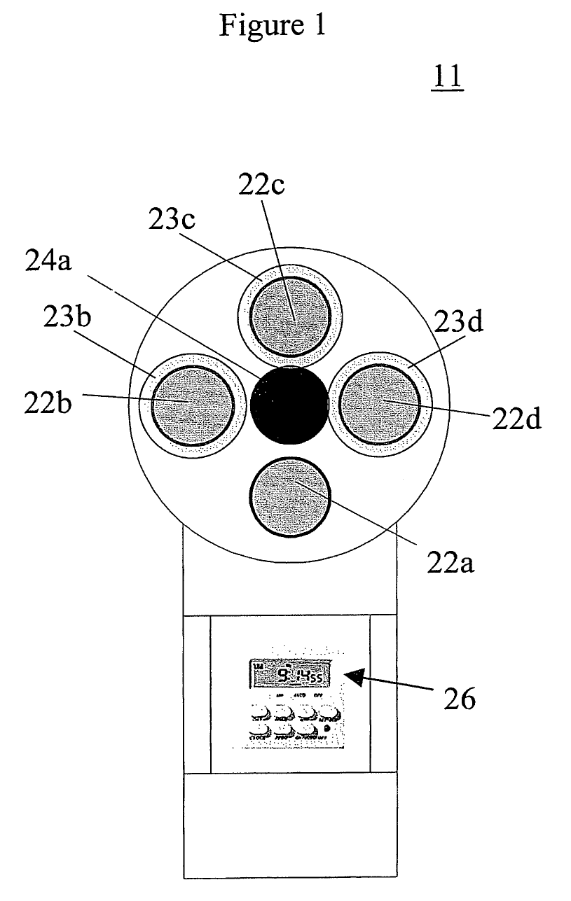

FIG. 1 illustrates a first embodiment 11 of a detector of internal tissue abnormalities according to the current invention. Embodiment 11 includes four pyroelectric IR sensors 22a-d that detect thermal waves (MIR radiation) coming from human body. Pyroelectric sensors 22a-d are based on the same principle as a thermo camera but operate at a wider spectral bandwidth (from 1 to 20-40 μm) than a thermal camera. Each sensor 22b-d has a band pass filter 23b-d respectively. Thus sensor 22a measures intensity of a wide band radiation signal (1-30 μm). Sensors 22b-d measure narrow band signals that pass through band pass filters 23b-d.

The use of a wide bandwidth allows the sensor 22a to accumulate energy radiated by human body, as a “black body” over a large bandwidth, and therefore detect weak signals from structures deep in the human body Particularly, the current invention facilitates finding anomalies (e g. cancerous lesions) inside the breast. By collecting radiation of wide collection bandwidth, sensor 22a also collects noises over a wide waveband coming from background and ambient obstructions. To increase the signal to noise ratio, the current invention employs contrast, a differential measure of radiation intensity, rather than interpreting measurements in terms of temperature differentials (as when using a thermal camera or FLIR according to the previous art). The advantages of contrast to detect small differences in radiation intensity is well known amongst those skilled in radio-astronomy [A. T. Nesmyanovich, V. N. Ivchenk, G P. Milinevsky, “Television system for observation of artificial aurora in the conjugate region during ARAKS experiments”, Space Sci. Instrument, vol 4, 1978, pp. 251-252. N. D. Filipp, V. N. Oraevskii, N. Sh. Blaunshtein, and Yu. Ya. Ruzhin, Evolution of Artificial Plasma Formation in The Earth's Ionosphere, Kishinev: Shtiintsa, 1986, 246 pages].

In the following embodiments of the current invention, contrast C is defined by the formula C=(R′−R″)/(R′+R″) where R′ is the overall heat flow from healthy tissue and R″ is the overall heat flow from the anomalous domain. For spectral measurements having different band widths the contrast is as above, but R′ and R″ are replaced by the spectral energy density R′(λᵢ) and R″(λᵢ). The mean spectral density of measured heat flows in each band of is computed according the formula S_{λi}=R(λᵢ)/Δλᵢ where S_{λi} is the mean spectral density of the heat flow for the chosen λᵢ band (ith waveband); R(λᵢ) is the measured value of the heat flow in the chosen λᵢ band; and Δλᵢ is the spectral width of the chosen ith band.

The spectral energy density radiated by a black body is given by the formulae

R″(λᵢ)=∫_{λni}^{λxi}[dR(λ,T)/dλ]{[εᵢₜ(λ)+ε_{can}(λ)]τ_{can}(λ)}dλ and

R′(λᵢ)=∫_{λni}^{λxi}[dR(λ,T)/dλ][εᵢₜ(λ)τᵢₜ(λ)]dλ where dR(λ,T)/dλ=kᵢλ⁻⁵[exp(k₂/λT)−1]⁻¹ and

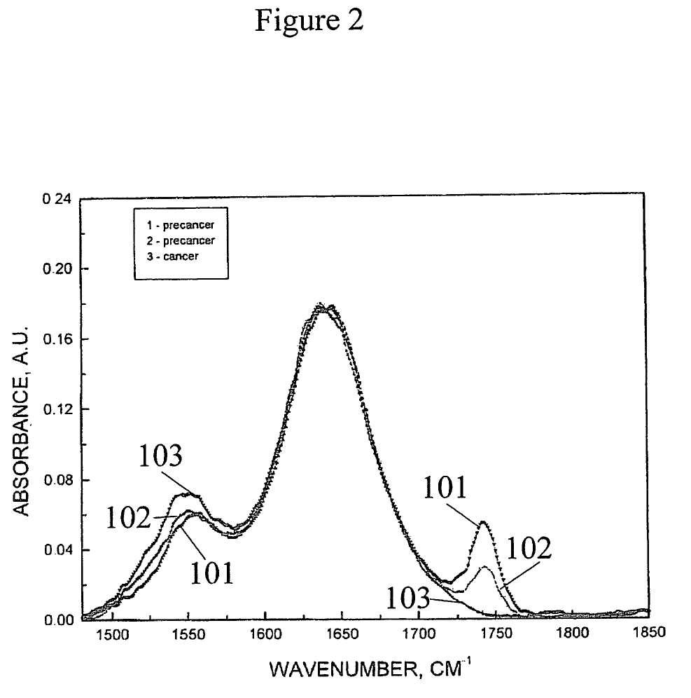

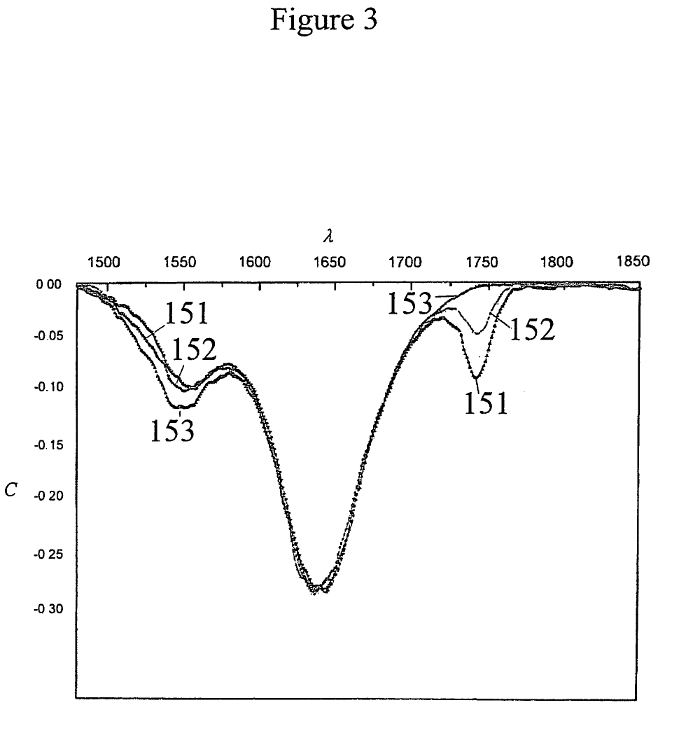

k₁=3.74×10⁻¹⁶ W×m⁴, k₂=1.44×10⁻² m×K; where dR(λ,T)/dλ is the special density of
heat flow from the black body at the temperature T (for living human tissue T=310° K); εᵢₜ is the heat radiation coefficient of blackness of normal living human tissue; τᵢₜ is the transparent coefficient of normal living human tissue; ε_{can} is the heat radiation coefficient of blackness of cancerous tissue; τ_{can} is the transparent coefficient of cancerous tissue. It is important to notice that the intensity of black body radiation is proportional to the blackness of the body. Thus, the intensity of light emitted by a body at a given waveband should be proportional to the absorbance in that waveband Since contrast is inversely proportional to emission intensity, therefore contrast of blackbody emittance is inversely proportional to absorbance as can be seen by comparing FIG. 3 to the absorbance data FIG. 2 from which FIG. 3 was computed (FIG. 2 is based on measurements made by Afanasyeva, N., S. Kolyakov, V. Letokliov, et al, “Diagnostic of cancer by fiber optic evanescent wave FTIR (FEW-FTIR) spectroscopy”, SPIE, vol. 2928, 1996, pp. 154-157. Afanasyeva, N., S. Kolyakov, V. Letokhov, and V. Golovkina, “Diagnostics of cancer tissues by fiber optic evanescent wave Fourier transform IR (FEW-FTIR) spectroscopy”, SPIE, vol. 2979, 1997, pp. 478-486 and Brooks, A., N. Afanasyeva, R. Bruch, et al., “FEW-FTIR spectroscopy applications and computer data processing for noninvasive skin tissue diagnostics in vivo”, SPIE, vol. 3595, 1999, pp. 140-151).

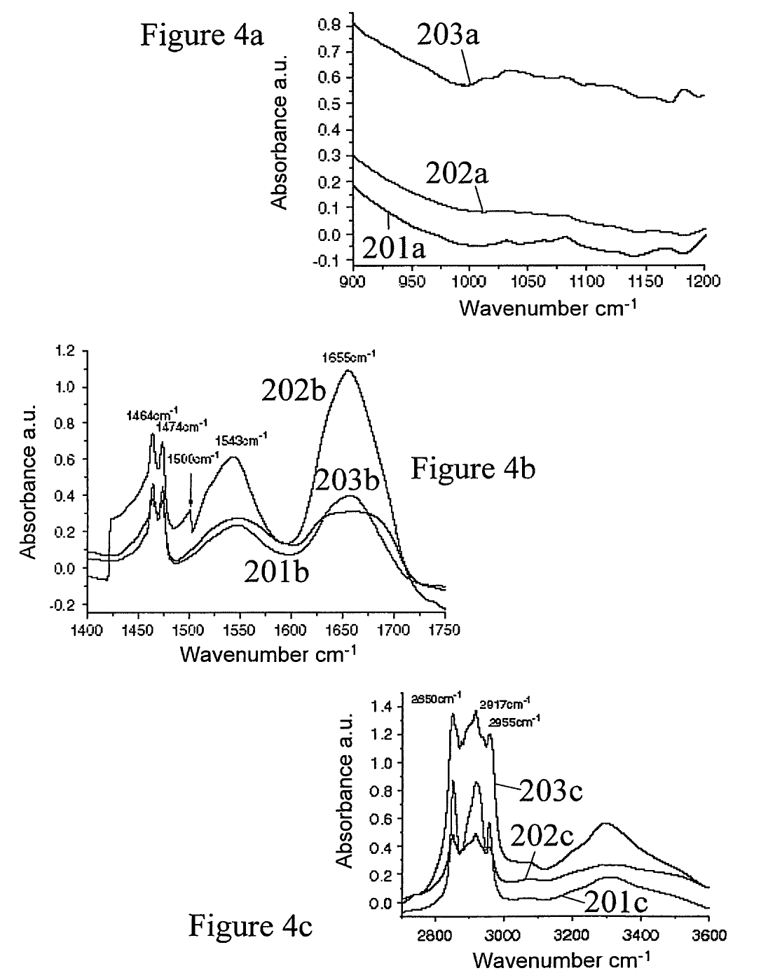

To increase signal strength and further increase the signal to noise ratio, the current invention employs an active method to preferentially heat lesions making them easier to detect. In the active method, lamp 24a, which is a MIR radiation source, heats the breast by irradiating the breast with MIR radiation in the frequency band of 1600-1700 cm⁻¹ at an intensity of 10 mW/mm². Alternatively, lamp 24a could also include a dimmer to allow heating with a lower intensity. Normal tissue does not absorb MIR radiation in the 1600-1700 cm⁻¹ (see FIG. 2a and FIG. 4b) thus light in this waveband passes through healthy tissue without heating the tissue. On the other hand, radiation in the 1600-1700 cm⁻¹ band is strongly absorbed by cancer tissue, (see FIG. 2a and FIG. 4b) and thereby heats cancerous tissue. Thus, radiation in the 1600-1700 cm⁻¹ preferentially heats cancerous lesions including lesions obscured behind healthy tissue but does not heat healthy tissue. FIG. 4a-c are based on measurements made by [Liu, C., Y. Zbang, X. Yan, X. Zhang, C. Li, W. Yang, and D. Shi, “Infrared absorption of human breast tissues in vitro”, J. of Luminescence, vol. 199-120, 2006, pp. 132-136.]

More specifically, lamp 24a is activated by a timer 26 for a predetermined period of 3 minutes. Irradiating the breast with light in the 1600-1700 cm⁻¹ wave band for 3 minutes heats the cancerous lesion without heating surrounding normal tissue. This increases the temperature differential between the cancerous lesion and surrounding normal tissue by approximately 0.3-1° K. The 0.3-1° K difference in the temperature between the cancerous lesions and healthy tissue causes an anomaly in black body thermal radiation that is large enough to be detected by existing pyroelectric detectors even under a few centimeters of healthy tissue.

After 3 minutes, timer 26 shuts down lamp 24a and activates sensors 22a-d. Then, an integral scan is made of the breast. Sensor 22a measures the integral signal in a wide waveband from 1-30 μm whereas sensors 22b-d measure signals in the narrow wavebands 1600-1700 cm⁻¹ (sensor 22a), 1000-1050 cm⁻¹ (sensor 22b), and 3250-3350 cm⁻¹ (sensor 22d). It can be seen in FIG. 4a-c that in the wave bands of sensors 22a -c with respect to normal tissue, cancerous lesions have much higher absorbance and precancerous lesions have slightly higher absorbance whereas in the waveband of sensor 22d, cancerous lesions have higher absorbance and precancerous lesions have less absorbance than normal tissue.

It can be seen from the above formula for computing R′(λᵢ) and R″(λᵢ) and from FIG. 2 and FIG. 3 that positive absorbance corresponds to negative contrast. Thus at the location of a cancerous lesion all four sensors 22a-d detect negative contrast and at the location of a precancerous lesion sensors 22a-c detect negative contrast whereas sensor 22d detects a positive contrast. It is emphasized that exposure to MIR radiation at a rate 10 mW/mM² for 3 minutes and heating breast tissue 1° K are harmless, painless and non-intrusive.

In order to decrease background noise measurements are made in a cool room and the exterior of the breast is stabilized in a plastic frame while the patient is in a prone position and the external tissue in the region of interest is cooled using fans.

FIG. 2 presents results of spectrographic analysis of IR energy absorbance by anomalous tissue structures, such as breast precancer 102, 103 and cancer 101. Precancer 102, 103 is an early and posteriori stage of the cancer evolution. According to results disclosed in Afanasyeva, et al, 1996; Afanasyeva, et al. 1997; and Brooks, et al. 1999. Based on FIG. 2 and the relations between the coefficients of absorbance, transparence, radiation and the contrast (as defined above), the calculated the contrast of the pre-cancer 152, 153 and cancer 151 tissues is presented in FIG. 3 [Liu, et al. 2006].

In both FIG. 2 and FIG. 3, it can be seen that pre-cancer 102, 103, 152, 153 and cancer 101, 151 have a maximum absorbance at ˜1630 cm⁻¹. Similarly results are seen in FIG. 4b [from Liu, et al. 2006] at 1655 cm⁻¹ for both precancer 203b and cancer 202b. Thus, as described above, radiation in a waveband near 1650 cm⁻¹ will pass through healthy breast tissue and heat precancerous and cancerous lesions. After heating, the lesions can be detected by black body MIR radiation emitted by the lesions due to their elevated temperature. Specifically, a one degree K temperature rise produces a MIR signal of ˜10⁻⁷-10⁻⁶ W/cm² at the skin surface (˜3 cm from the lesion) which can easily, dependably and accurately be detected by a commonly available pyroelectric detector.

The present invention takes advantage of spectral differences in the absorbance and emittance of MIR radiation to differentiate between benign lesions from malignant lesions. Particularly, as illustrated in FIG. 4c at 3300 cm⁻¹ breast cancer 203c absorbs more strongly than normal tissue 201c whereas precancerous lesions 202c absorb MIR light in the 3000 cm⁻¹ waveband less than normal tissue 201c. Thus according to the formula above the contrast of blackbody radiation from cancer 203c at 3300 cm⁻¹ is negative and the contrast of blackbody radiation from a precancerous lesion 202c at 3300 cm⁻¹ is positive.

Alternatively, according to FIG. 3 the contrast of a cancerous lesion 153 at −1750 cm⁻¹ is nearly zero whereas the contrast for a precancerous lesion 151, 152 is positive. Also according to FIG. 4b at 1750 cm⁻¹ the absorbance of a precancerous lesion 202b is greater than the absorbance of normal tissue 201b whereas the absorbance of a malignant lesion 203b is less than the absorbance of normal tissue 201b. This fact can be used for differentiation in earlier stage of diagnostics the pre-cancer and the cancer structures.

Alternatively, different types of lesions can be differentiated by their absorbance directly. Thus, when the breast heated by radiation having wavenumber near 1650 cm⁻¹, both cancerous 103, 153, 203b and benign lesion 102, 101, 151, 152, 202b will be heated and therefore will be detected as hot spots in a wide band MIR integral scan whereas when the breast is heated by radiation having wavenumber near 1550 cm⁻¹ only cancerous lesions 103, 153, 203b will be heated. Thus those lesions 103, 153, 203b detected both after heating at 1550 cm⁻¹ and 1650 cm⁻¹ are identified as malignant whereas those lesions 102, 101, 151, 152, 202b which are apparent in an integral scan after heating at 1650 cm⁻¹ but are not apparent after heating at 1550 cm⁻¹ are identified as benign.

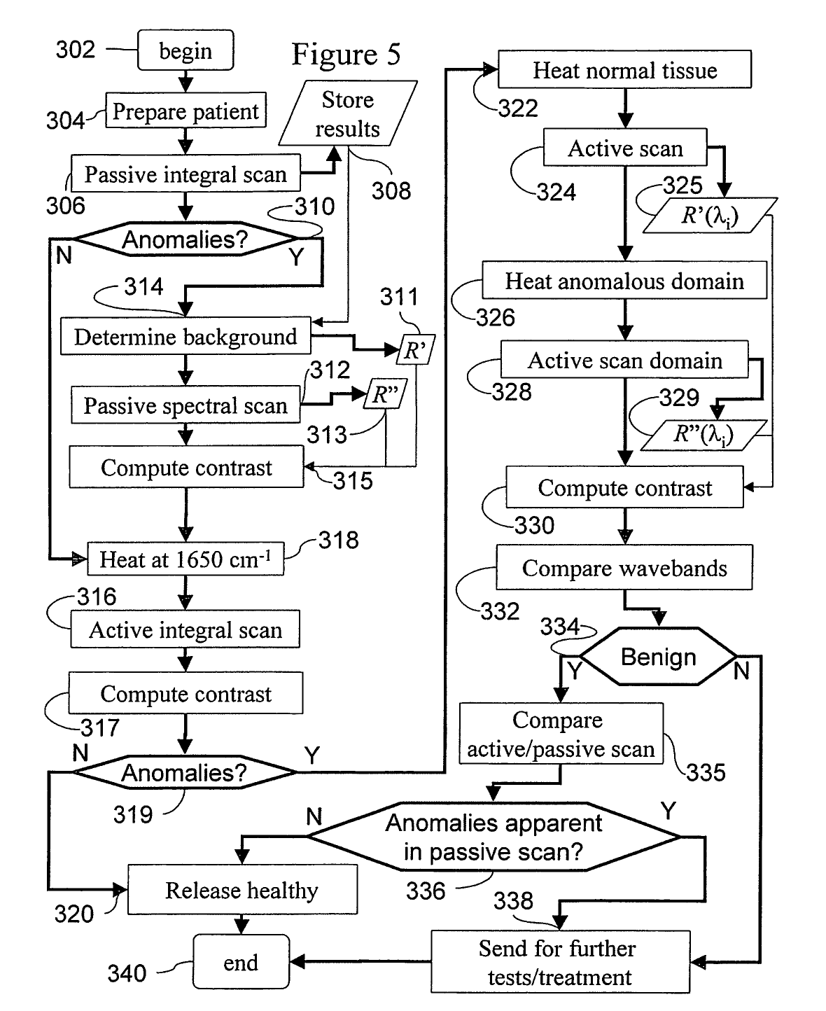

FIG. 5 is a flow chart of a first embodiment of the current invention. In the embodiment of FIG. 5 differential heating due to differential absorption of MIR energy is used to differentiate both precancerous lesions and cancer from healthy breast tissue while spectral differences in emittance is used to differentiate between malignant and benign lesions. At the start 302 of a diagnostic session the patient is prepared 304 for the exam. The exam takes place in a cool room and the external portion of area to be examined is kept cool by a fan blowing cool air. The patient is positioned in order that the region to be scanned remains as still as possible (for example in a prone position as described in Harrison et al. 1999 U.S. Pat. No. 5,999,842). A passive integral scan 306 is performed. Preferentially, the detector of FIG. 1 is used for scanning. For the passive integral scan 306, lamp 24a remains off.

During integral scan 306, sensor 22a measures over a wide waveband 333-10,000 cm⁻¹ while simultaneously sensors 22b-d measure narrow wavebands 1600-1700 cm⁻¹ (sensor 22a), 1000-1050 cm⁻¹ (sensor 22b), and 3250-3350 cm⁻¹ (sensor 22d). The results 308 are stored. If domains of anomalous heat flow are identified 310 in passive integral scan 306 then those zones are further tested at a higher detail in a passive spectral scan 312. In order to perform the passive spectral scan 312, a background heat flow (R′ 311) is determined 314 from a passive integral scan results 308 by averaging the radiation intensity over areas where no anomalous flow was observed for each spectral waveband measured by sensors 12a-d. Then the spectral scan 312 is performed and R″ 313 is measured in domains displaying anomalous heat flow in passive integral scan 306. During passive spectral scan 312, detector 11 is held over the scanned domain for a longer time than during integral scan 306 (averaging over a longer time reduces transient noise) Also during passive spectral scan 312, detector 11 is held as close as possible to the skin of the scanned domain and the anomaly is scanned from various angles to get a three dimensional picture of the anomalous domain including the depth under the skin surface. Using equations above, contrast C is computed 315 in the domain of anomalous flow.

Alternatively, to get more spectral detail, the detector of FIG. 1 is used for the integral scan, but the spectral scan is made using a full spectrum methodology (for example FTIR). Alternatively, when spectral detail is of less interest, the integral scan can be done for one waveband only and the multiple wavebands are measured only in the detailed spectral scan.

If no anomalies of heat flow had been detected 310 in passive integral scan 306, then passive spectral scan 312-315 would be skipped.

After passive scan 306-315 an active integral scan 316 is performed. To perform active integral scan 316, first the entire region of interest is exposed 318 to MIR radiation in the waveband of 1600-1700 cm⁻¹ at an intensity of 10 mW/mm² for 3 minutes using heat lamp 24a (while still cooling the surface of the region using cool air and fans as above). MIR radiation in the frequency band of 1600-1700 cm⁻¹ preferentially penetrates normal tissue and heats cancerous and precancerous lesions as can be seen in FIG. 2, FIG. 3, and FIG. 4b. After 3 minutes heat lamp 24a is deactivated and active integral scan 316 is performed. Active integral scan 316 is performed exactly like passive integral scan 306-315, but because exposure 318 increased the temperature differential between lesions and normal tissue, active integral scan 316 is much more sensitive that passive integral scan 312. The determination of anomalous zones, background radiation levels and contrast 317 is exactly similar to the passive integral scan (306-315 above).

If no domains of anomalous heat flow are observed 319 neither in passive integral scan 312 nor in the active integral scan 316, then the patient is diagnosed 320 as free of detectable lesions and the session ends 340.

If domains of anomalous beat flow are observed 319 either in passive integral scan 306 or in active integral scan 316, then the domains of anomalous flow are tested by performing an active spectral scan 328. In order to perform active spectral scan 328, first the background spectral intensity R′(λᵢ) 325 must be determined by actively scanning 324 a few areas without anomalies. In the example of FIG. 5, the heat flow anomaly found in the integral scan is very weak Therefore while analyzing the results of the integral scan, it is determined that in order to increase the sensitivity of the spectral scan, timer 26 will be set for a predetermined heating period of (5 min), which is longer than the heating period of the active integral scan (3 minutes). MIR radiation from lamp 24a is well below the intensity that would endanger or discomfort the patient Nevertheless, it is undesirable to expose the patient to heating for long periods. Thus, for the initial scans when there was no reason to suspect a lesion, minimal exposure took priority over sensitivity and only 3 minutes of exposure were used in the case where there is a suspected lesion, it is deemed worthwhile to use a higher level of heating to increase the sensitivity of the test In order to determine the background radiation levels for active spectral scan 328, a few areas where no anomaly was found are heated 322 locally using lamp 24a for 5 minutes and scanned 324 in each of the spectral wavebands (λ₁=333-10,000 cm⁻¹, λ₂=1600-1700 cm⁻¹, λ₃=1000-1050 cm⁻¹, and λ₄=3250-3350 cm⁻¹). The scan results in a few normal locations are averaged to determine background levels R′(λᵢ) 325 for each of the active spectral bands λᵢ. Averaging helps reduce local noise effects.

After determining the background radiation level R′(λᵢ) 325 for each waveband λᵢ for the longer heating period (5 minutes) of spectral scan 328, then the domains of identified anomalies are heated 326 for 5 minutes by lamp 24a. After heating 326, the anomalous domains are scanned 328 to determine the local active spectral radiation intensity R″(λᵢ) 329 The active spectral results R′(λᵢ) 325 and R″(λᵢ) 329 ate used to compute contrast 330.

Analysis of results starts by comparing 332 the results on different wavebands to determine 334 if the detected lesions are benign. If contrast C(λᵢ)=[R′(λᵢ)-R′(λᵢ)]/[R′(λᵢ)+R′(λᵢ)] is negative for i=1,2,3 and positive for i=4 and the spectral contrast (comparing emittance in two wavebands at one locations) between wave bands 2 and 3 ([R″(λ₂)-R″(λ₃)]/[R″(λ₂)+R″(λ₃)]) is less than 0.5 then the domain is determined 334 to be benign lesion. Otherwise, the domain is not determined 334 to be a benign lesion and the patient is sent for further testing and treatment.

It should be noted that the embodiment of FIG. 5 allows spectral scanning to identify various lesions quickly (heating the breast once for each scan and not requiring a cooling off period between scans). Nevertheless, in the embodiment of FIG. 5 there is a possible confounding effect in the spectral results. Particularly, MIR radiation in the waveband 1600-1700 cm⁻¹ heats tumor precursors to a higher temperature than surrounding tissue. Also in the passive regime cancer precursors are often hotter than healthy tissue due to increased metabolic activity. Therefore, even though (as shown in FIG. 4c) for lesions and healthy tissue at the same temperature, the emittance of precancerous lesions at 3300 cm⁻¹ is less than the emittance of healthy tissue, nevertheless at elevated temperatures lesions may emit more radiation in this bandwidth than cooler healthy tissue. Therefore the negative contrast shown in FIG. 4c may not be observable in the example of FIG. 5. While this difficulty is somewhat lessened using spectral contrast (comparing emittance in two different wavebands at a single location (e.g. C(λₘ,λₙ)=[R″(λₘ)-R″(λₙ)]/[R″(λₘ)+R″(λₙ)]), it may sometimes be difficult to differentiate between benign and malignant tumors using the methodology of FIG. 5.

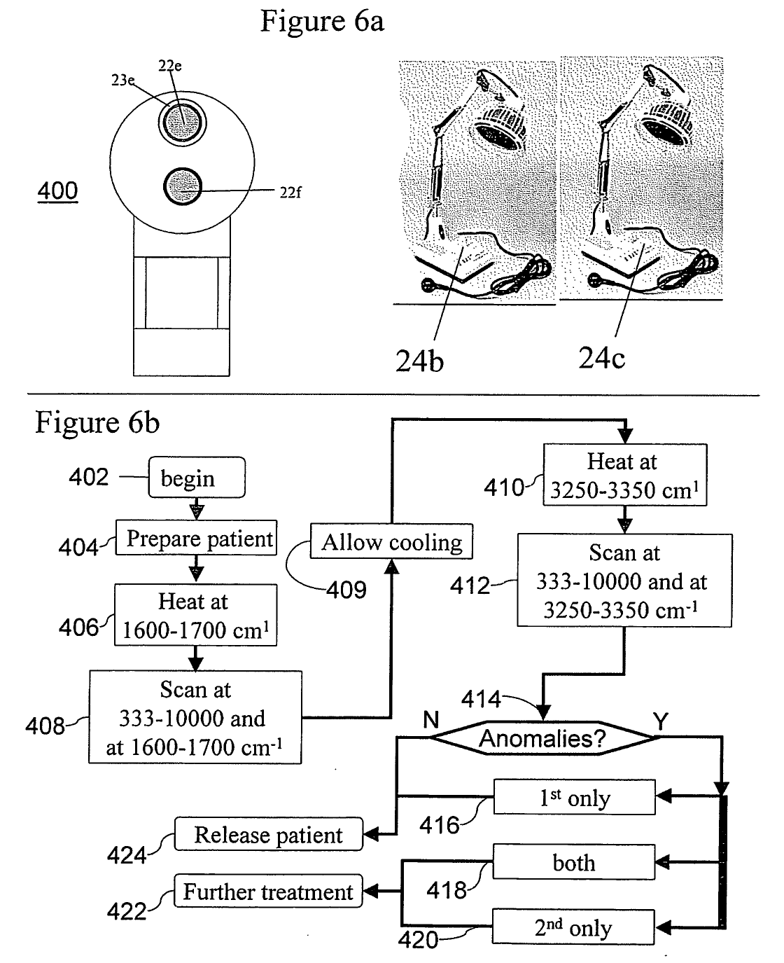

If all of the lesions observed 319 are determined 334 to be benign, then the active and passive results are compared 335 if none of the lesions are found 336 large enough to be identified 310 in the passive integral scan then the patient is declared healthy and released. If all of the lesions observed 319 are determined 334 to be benign, but some of the lesions are found 336 large enough to be identified 310 in the passive integral scan then the patient is sent for further tests 338. Further testing may include more careful rescanning anomalous domains, including scanning after heating with MIR illumination of various wavebands (see FIG. 6a,b and associated discussion) or other tests known in the art.

A second alternative embodiment of the invention of the current patent is illustrated in FIG. 6a,b. In the embodiment of FIG. 6a,b differences in heating due to differential absorption of MIR energy as well as differences in emissivity are used to differentiate among healthy breast tissue, malignant lesions and benign lesions. Thus, the embodiment of FIG. 6a,b may be used for further testing in cases where a preliminary test according to the embodiment of FIG. 5 gives ambiguous results.

FIG. 6a shows a second embodiments of system to identify lesions inside the breast of a patient. The system includes two MIR lamps. A first lamp 24b radiates energy in a first waveband 1600-1700 cm⁻¹ and a second lamp 24c radiates energy in a second waveband 3250-3350 cm⁻¹. The system also includes a detector 400 with two pyroelectric sensors 22e and 22f sensitive to MIR radiation in the waveband from 333-10,000 cm⁻¹ and an interchangeable band pass filter 23e. Thus detector 400, scans simultaneously on a wide waveband 333-10000 cm⁻¹ and on and adjustable waveband.

FIG. 6b is a flow chart illustrating a second embodiments of system to identify lesions inside the breast of a patient. The method begins 402 by preparing the patient 404 (preparations are similar to those described in FIG. 5 step 304). The region to be scanned is then heated 406 by MIR radiation in a first waveband 1600-1700 cm⁻¹ at an intensity of 10 mW/mm² for 3 minutes using heat lamp 24b. MIR radiation in the first waveband is absorbed preferentially by both tumors and benign lesions. The region is then scanned 408 using detector 400 with a 1600-1700 cm⁻¹ exchangeable filter 23c. Thus the region is scanned 408 simultaneously over a wide waveband 333-10000 cm⁻¹ receiving a large portion of the available energy (getting the strongest possible signal) and over the band 1600-1700 cm⁻¹ which is the waveband that should be most strongly indicative of lesions (getting the best signal to noise ratio).

The region is then allowed to cool 409 back to equilibrium. Allowing the region to cool 409 takes time adding to the inconvenience of the procedure, but if precancerous domains were not allowed to cool, they would be hard to differentiate from malignant domains in the next step. After the region reaches equilibrium, the region is heated 410 by exposure to MIR radiation in a second waveband, 3250-3350 cm⁻¹, at an intensity of 10 mW/mm² for 3 minutes using heat lamp 24c. MIR radiation in the second waveband is absorbed preferentially by tumors and is not absorbed by benign lesions. The region is then scanned 412 using detector 400 using a 3250-3350 cm⁻¹ exchangeable filter 23e. Thus the region is scanned 412 simultaneously over a wide waveband 333-10000 cm⁻¹ receiving a large portion of the available energy (getting the strongest possible signal) and over the band 3250-3350 cm⁻¹, which is the waveband that should be most strongly indicative of malignant lesions (getting the best signal to noise ratio).

If no anomalies are found 414 then the patient is found clear of suspicious lesions and released If anomalies are found 414 then if the anomalous domains emit higher than normal MIR radiation the first 408 scan but not in the second scan 412, the lesions are declared 416 benign and the patient released 424 with follow up to make sure that the benign lesions do not become cancerous. On the other hand, if higher than normal emittance was found 414 in at least one domain in both the first scan 408 and the second scan 412 then the lesions are assumed 418 malignant and the patient is sent for further testing and treatment 422. Similarly if additional emittance is found 414 in the second scan 412 but not the first scan 408 then the test is declared inconclusive 420 and the patient is sent for further testing 412 to determine what kind of lesions she does have.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A non-intrusive method for identifying an anomalous domain under a skin in a region of a patient, comprising the steps of:
a) heating the anomalous domain preferentially over healthy tissue;
b) measuring an emitted radiation from the anomalous domain as a result of said beating; and
c) detecting the anomalous domain based on a result of said measuring.

2. The method of claim 1, wherein said step of heating includes applying infrared radiation in a first waveband to the region.

3. The method of claim 2, wherein said first waveband differs from a wave band of said emitted radiation.

4. The method of claim 2, further comprising the step:
d) applying an infrared radiation in a second waveband to the region.

5. The method of claim 2, wherein first waveband includes infrared radiation having a wave number 1600-1700 cm⁻¹.

6. The method of claim 1, wherein said emitted radiation includes a black body radiation in a medium infrared waveband.

7. The method of claim 1, wherein the region includes a portion of the breast of the patient.

8. The method of claim 1, wherein said step of heating is for a predetermined period of time and said step of measuring occurs after said period of time.

9. The method of claim 1, wherein said result is a differential measure of said emitted radiation.

10. The method of claim 9, wherein said differential measure is a contrast.

11. The method of claim 1, further comprising the step:
d) performing a spectral analysis to identify the anomalous domain.

12. The method of claim 1, further comprising the step:
d) determining a depth of the anomalous domain.

13. A detector to reveal an anomalous domain under a skin of a region of a patient comprising:
a) a lamp configured to beat the region by exposing the skin to MIR radiation;
b) a timer for turning off said lamp after a predetermined period of exposure; and
c) a MIR sensor for measuring a radiation emitted from the region after heating with said lamp.

14. The detector of claim 12, further comprising:
d) a band pass filter to limit the sensitivity of said MIR sensor to a first narrow waveband.

15. The detector of claim 14, further comprising:
e) A second MIR sensor for measuring radiation in a second waveband.
